(19) United States
(12) Patent Application Publication
(10) Pub. No.: US 2013/0189162 A1
(43) Pub. Date: Jul. 25, 2013

(54) PHOTOCATALYST UNIT

(71) Applicant: Cheolgyu Jeong, Busan (KR)

(72) Inventor: Cheolgyu Jeong, Busan (KR)

(73) Assignee: SMARTECH CO., LTD., Busan (KR)

(21) Appl. No.: 13/515,704

(22) PCT Filed: Jul. 15, 2011
(86) PCT No.: PCT/KR11/05226

§ 371 Date: Jun. 13, 2012

(30) Foreign Application Priority Data
May 26, 2011 (KR) 10-2011-0050068

(51) Int. Cl. A61L 9/20

(52) U.S. Cl. CPC A61L 9/205
USPC 422/121

(57) ABSTRACT

The present invention relates to a photocatalyst unit. More particularly, the photocatalyst unit includes a body made of a plate with openings at upper and lower sides thereof and having one or more pairs of opposing lamp fixing means provided therein; ultraviolet radiation lamps each coupled to the lamp fixing means; a photocatalyst member coupled to the body and having a surface coated with a photocatalyst, wherein the photocatalyst member includes a plurality of plates disposed orthogonally to the ultraviolet radiation lamps, wherein the plates are disposed parallel to a flow direction of air and spaced to each other in order to form gaps between adjacent plates; and photocatalyst filters each disposed on upper and lower sides of the photocatalyst member and mounted on the body to cover upper and lower sides of the body, wherein the photocatalyst filters are applied with a photocatalyst.

### TECHNICAL FIELD

The present invention relates, in general, to a photocatalyst unit, and more particularly to a photocatalyst unit in which ultraviolet radiation emitted from an ultraviolet radiation lamp is maximizedly irradiated on the surrounding photocatalyst members and photocatalyst filters to increase an ultraviolet radiation reaction area.

### BACKGROUND ART

Typically, a photochemical reaction caused by a combination of a photocatalyst and ultraviolet radiation provides excellent sterilizing and deodorizing actions, and thus is used for air cleaning and sterilizing. In particular, the photocatalyst is a useful technology in that the photocatalyst can be semipermanently used, be harmless to humans to be stably employed, and don't cause a secondary contaminant in contrast to other contamination prevention technologies.

However, when the ultraviolet radiation is irradiated on photocatalyst bodies, the ultraviolet radiation is blocked by a front photocatalyst body not to reach the remaining rear portions. Accordingly, various methods have been found to maximize the irradiation amount of ultraviolet radiation on the photocatalyst bodies by combining the photocatalyst with the ultraviolet.

In general, such photochemical reaction technique using the photocatalyst essentially provides sterilizing and cleaning of air by directly contacting a surface of the photocatalyst with contaminated air. If a contact area between the air and the activated photocatalyst layer is small, then the cleaning efficiency is decreased. On the contrary, if the contact area is increased, air flow paths become narrow, thereby increasing the pressure loss.

Therefore, there is required an apparatus which can have a greater contact area with air and a lower pressure loss. Accordingly, various techniques are proposed in which photocatalyst bodies are coupled to ultraviolet lamps in various configurations.

Firstly, a configuration, in which opposing surfaces made of a mesh body constitute photocatalyst bodies and ultraviolet radiation lamps are installed between the photocatalyst bodies, is known. In this case, because light is irradiated on only two flat surfaces, the pressure loss is low, but the irradiation area of ultraviolet radiation is very small.

In addition, for a structure like a honeycomb, a photocatalytic action is activated only on an inlet portion thereof irradiated by ultraviolet radiation, and the ultraviolet radiation cannot reach the inside thereof, thereby decreasing efficiency of photo oxidation reaction. Accordingly, a surface area carrying the photocatalyst is greater, but regions on which the ultraviolet radiation is not irradiated are caused, thereby decreasing deodorizing efficiency.

### DISCLOSURE

### Technical Problem

Accordingly, the present invention has been made keeping in mind the above problems occurring in the related art, and an object of the present invention is to provided a photocatalyst unit in which a contact area between ultraviolet radiation and photocatalyst bodies can be increased and the ultraviolet radiation can be uniformly irradiated to achieve excellent photo oxidation reaction.

Another object of the present invention is to provide a photocatalyst unit which can occupy a minimized space to achieve a thin and compact structure.

A further object of the present invention is to provide a photocatalyst unit in which a smooth air flow can be generated without causing the pressure loss of air, and a contact area of air with photocatalyst filters and photocatalyst members can be increased, thereby increasing sterilizing and deodorizing efficiencies.

### Technical Solution

In order to achieve the above objects, there is provided a photocatalyst unit, including: a body made of a plate with openings at upper and lower sides thereof and having one or more pairs of opposing lamp fixing means provided therein; ultraviolet radiation lamps each coupled to the lamp fixing means; a photocatalyst member coupled to the body and having a surface coated with a photocatalyst, wherein the photocatalyst member includes a plurality of plates disposed orthogonally to the ultraviolet radiation lamps, wherein the plates are disposed parallel to a flow direction of air and spaced to each other in order to form gaps between adjacent plates; and photocatalyst filters each disposed on upper and lower sides of the photocatalyst member and mounted on the body to cover upper and lower sides of the body, wherein the photocatalyst filters are applied with a photocatalyst.

In this case, the photocatalyst member may be coupled to the body on upper and lower sides of the ultraviolet radiation lamps, and may have semi-circular grooves formed therein for each receiving the ultraviolet radiation lamps.

Also, the plates may have fastening holes formed on both ends thereof, and fixing bars may be extended through and coupled to the fastening holes, such that the plurality of plates can be integrated.

Furthermore, spacer members may be provided to be coupled to the fixing bars between adjacent plates for constantly keeping distances between adjacent plates.

In addition, the body has receiving grooves formed on upper and lower ends thereof, and the photocatalyst filters are inserted into and removed from the receiving grooves.

### Advantageous Effects

According to the present invention, the following effects may be obtained.

Firstly, the ultraviolet radiation can be stereoscopically irradiated all over the photocatalyst member and the photocatalyst filters such that irradiation efficiency of the light beam can be increased and a photocatalytic reaction area can be increased.

Also, the ultraviolet radiation from the ultraviolet radiation can uniformly reach the photocatalyst.

Also, the photocatalyst member can be formed parallel to the flow direction of air, such that the pressure loss of air flowing through air flow paths formed by gaps within the photocatalyst member can be decreased, thereby achieving a smooth air flow.

In addition, a space occupied by the photocatalyst unit can be reduced, and thus can be easily assembled to or dissembled from various applications requiring the photocatalyst unit, such as an air-conditioner or a refrigerator.

Furthermore, the photocatalyst member, the photocatalyst filters, and the ultraviolet lamps can be easily replaced or repaired.

### DESCRIPTION OF DRAWINGS

FIG. 1 is a perspective view of a photocatalyst unit according to the present invention;

FIG. 2 is an exploded perspective view of the photocatalyst unit according to the present invention;

FIG. 3 is a sectional view taken along a line B-B′ of an embodiment of the photocatalyst unit according to the present invention; and

FIG. 4 is a sectional view taken along a line A-A′ of the photocatalyst unit according to the present invention.

### BEST MODE

According to an exemplary embodiment of the present invention, there is provided a photocatalyst unit, including a body made of a plate with openings at upper and lower sides thereof and having one or more pairs of opposing lamp fixing means provided therein; ultraviolet radiation lamps each coupled to the lamp fixing means; a photocatalyst member coupled to the body and having a surface coated with a photocatalyst, wherein the photocatalyst member includes a plurality of plates disposed orthogonally to the ultraviolet radiation lamps, wherein the plates are disposed parallel to a flow direction of air and spaced to each other in order to form gaps between adjacent plates; and photocatalyst filters each disposed on upper and lower sides of the photocatalyst member and mounted on the body to cover upper and lower sides of the body, wherein the photocatalyst filters are applied with a photocatalyst.

### Exemplary Embodiments

Exemplary embodiments of the present invention will now be described in detail with reference to the accompanying drawings.

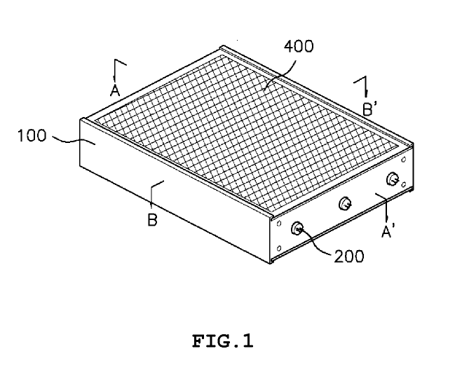

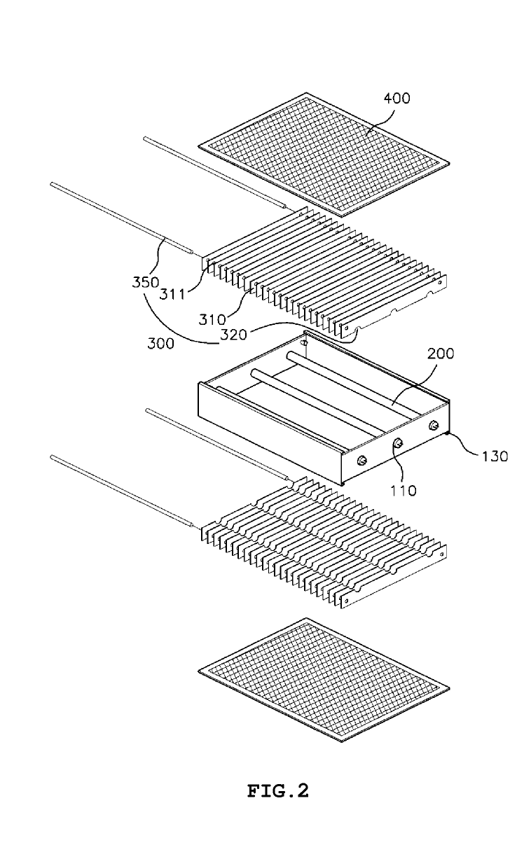

FIG. 1 is a perspective view of a photocatalyst unit according to the present invention, and FIG. 2 is an exploded perspective view of the photocatalyst unit according to the present invention.

As shown in FIGS. 1 and 2, the present invention is constituted of a body 100, and ultraviolet radiation lamps 200, photocatalyst members 300 and photocatalyst filters 400.

The body 100 is made of a plate with openings at upper and lower sides thereof and has one or more pairs of opposing lamp fixing means 110 provided therein. The ultraviolet radiation lamps 200 are each coupled to the lamp fixing means 110 such that one or more ultraviolet radiation lamps 200 are arranged in the body 100. In this case, the body 100 has preferably a mounting means (not shown) formed thereon such that the body 100 can be easily mounted on an air-conditioner or other air cleaning apparatuses, and the ultraviolet radiation lamps 200 has preferably a width corresponding to that of photocatalyst members 300 as described below. In addition, a terminal for connecting to an extender power supply is installed on one end of the ultraviolet radiation lamps 200.

In this time, the lamp fixing means 110 may be embodied by forming holes in the plate of the body, or may be configured to fix the ultraviolet radiation lamps 200 in other various known manners.

The photocatalyst members 300 are coupled to the body 100 on upper and lower sides of the ultraviolet radiation lamps 200 and have a surface coated with a photocatalyst such that, upon irradiating, the ultraviolet radiation causes a photochemical reaction with the coated photocatalyst. The photocatalyst members 300 may be made of various materials, such as papers, PVCs, non-woven fabrics, metal pieces, or glasses. Also, the photocatalyst members 300 are preferably constituted of flat plates, although any materials allowing air to pass therethrough, such as mesh materials, filters, or porous materials, may be employed.

Also, a plurality of plates 310, which are disposed parallel to a flow direction of air and spaced to each other in order to form gaps between adjacent plates, disposed orthogonally to the ultraviolet radiation lamps 200. Because the ultraviolet radiation is irradiated into the gaps between adjacent plates 310, the ultraviolet radiation is irradiated all over the photocatalyst members 300 without causing shadows such that regions, on which the ultraviolet radiation is not irradiated, are eliminated, thereby increasing sterilizing and deodorizing efficiencies.

Furthermore, because the plurality of plates 310 of the photocatalyst members 300 are disposed parallel to the flow direction of air, any pressure loss is not caused and a contact area between air and the photocatalyst members 300 is maximized.

The photocatalyst filters 400 are each disposed on upper and lower sides of the photocatalyst member 300 and mounted on the body 100 to cover upper and lower sides of the body 100. Also, the photocatalyst filters 400 are applied with a photocatalyst.

In this case, the photocatalyst filters 400 may be made of any materials, such as metal filters, paper filters, PVC filters, non-woven fabrics, glass fibers, or any combinations thereof, in a form of a net-shaped body, a porous body, or a mesh body. Also, the photocatalyst filters 400 respectively cover one surface of each of the photocatalyst members 300 and thus react with the ultraviolet radiation irradiated into the gaps between adjacent plates 310.

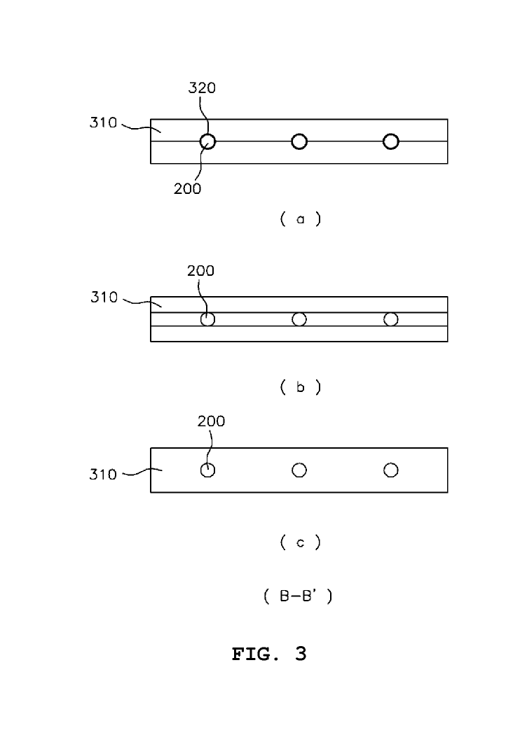

FIG. 3 is a sectional view taken along a line B-B′ of an embodiment of the photocatalyst unit according to the present invention.

As shown in FIGS. 2 and 3, the photocatalyst members 300 have semi-circular grooves 320 formed therein for each receiving and surrounding the ultraviolet radiation lamps 200. As a result, a thickness of the body 100 can be decreased, thereby reducing occupying space thereof.

Meanwhile, instead of forming the semi-circular grooves 320 as described above, the plates 310 may be formed to have a flat bottom surface as shown in FIG. 3(b), or the ultraviolet radiation lamps 200 may be extended through the plates of the photocatalyst member 300 to be integrally formed with the photocatalyst member 300 as shown in FIG. 3(c).

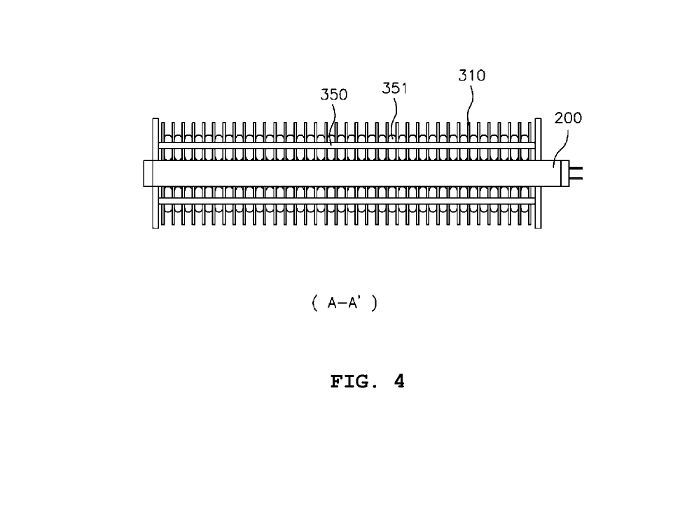

FIG. 4 is a sectional view taken along a line A-A′ of the photocatalyst unit according to the present invention.

As shown in FIGS. 2 and 4, the plates 310 have fastening holes 311 formed on both ends thereof, and fixing bars 350 are extended through and coupled to the fastening holes 311, such that the plurality of plates 310 are integrated. In addition, the body 100 may further have coupling means (not shown) on the inside thereof for releasably attaching the fixing bars 350. However, if the fixing bars 350 can releasably attached, any manners can be used at the discretion of those skilled in the art.

Meanwhile, spacer members 351 may further provided to be coupled to the fixing bars 350 for constantly keeping distances between adjacent plates 310. Preferably, the spacer members 351 may be formed in a shape capable of minimizing blocking of the ultraviolet radiation.

Also, the body 100 has receiving grooves 130 formed on upper and lower ends thereof, and the photocatalyst filters 400 are inserted into and coupled to the receiving grooves 130, such that the photocatalyst filters 400 can be removed upon replacing or repairing.

Now, a method for manufacturing the present invention configured as above will be described with reference to FIGS. 1 to 4.

Firstly, the ultraviolet radiation lamps 200 are coupled inside the body 100 such that power supply terminals can be connected to the outside. Then, the plates 310 and spacer members 351 are inserted onto the fixing bars 350 in an alternating fashion. The resulting photocatalyst members 300 are each disposed on upper and lower sides of the ultraviolet radiation lamps 200 such that photocatalyst members 300 are arranged orthogonally to the ultraviolet radiation lamps 200. In this time, the plates 310 are importantly disposed parallel to an air flow such that the pressure loss of air can be decreased.

Next, the photocatalyst filters 400 are inserted into the receiving grooves 130 of the body 100 such that the body 100 can be closed. The photocatalyst unit manufactured through the above procedures can be mounted on various applications, including a vehicle air-conditioner, an indoor air-conditioner, an air cleaning apparatus and the like.

As set fourth above, an essential technical spirit of the present invention is to provide a photocatalyst unit in which ultraviolet radiation can be stereoscopically irradiated all over photocatalyst members and photocatalyst filters such that irradiation efficiency of the light beam can be increased and a photocatalytic reaction area can be increased, thereby enhancing an air cleaning function. Accordingly, those skilled in the art will appreciate that various modifications can be made without departing from the scope of the essential technical spirit of the invention as described above.

### INDUSTRIAL APPLICABILITY

The present invention can be widely applied in products related to air cleaning and sterilization, which require excellent sterilizing and deodorizing actions.

What is claimed is:

1. A photocatalyst unit, comprising:
a body made of a plate with openings at upper and lower sides thereof and having one or more pairs of opposing lamp fixing means provided therein;
a plurality of ultraviolet radiation lamps each coupled to the lamp fixing means;
a photocatalyst member coupled to the body and having a surface coated with a photocatalyst, wherein the photocatalyst member includes a plurality of plates disposed orthogonally to the ultraviolet radiation lamps, wherein the plates are disposed parallel to a flow direction of air and spaced to each other in order to form gaps between adjacent plates; and
a plurality of photocatalyst filters each disposed on upper and lower sides of the photocatalyst member and mounted on the body to cover upper and lower sides of the body, wherein the photocatalyst filters are applied with a photocatalyst.

2. The photocatalyst unit according to claim 1, wherein the photocatalyst member is coupled to the body on upper and lower sides of the ultraviolet radiation lamps, and has semi-circular grooves formed therein for each receiving the ultraviolet radiation lamps.

3. The photocatalyst unit according to claim 1, wherein the plates have fastening holes formed on both ends thereof, and fixing bars are extended through and coupled to the fastening holes, such that the plurality of plates are integrated.

4. The photocatalyst unit according to claim 3, further comprising spacer members formed to be coupled to the fixing bars between adjacent plates for constantly keeping distances between adjacent plates.

5. The photocatalyst unit according to claim 1, wherein the body has receiving grooves formed on upper and lower ends thereof, and the photocatalyst filters are inserted into and removed from the receiving grooves.
